(19) United States
(12) Patent Application Publication
(10) Pub. No.: US 2005/0197640 A1
(43) Pub. Date: Sep. 8, 2005

(54) Portable battery operated aspirator

(76) Inventor: Samuel L. Say, La Canada, CA (US)

Correspondence Address: CHRISTIE, PARKER & HALE, LLP, PO BOX 7068, PASADENA, CA, 91109-7068, US

(21) Appl. No.: 11/035,387

(22) Filed: Jan. 13, 2005

Related U.S. Application Data
(63) Continuation-in-part of application No. 10/376,496, filed on Feb. 28, 2003.
(60) Provisional application No. 60/360,289, filed on Feb. 28, 2002.

(51) Int. Cl. A61M 1/00

(52) U.S. Cl. 604/319

(57) ABSTRACT

A portable aspirator has a canister, a housing that removably receives the canister, and a rigid catheter directly connected to the canister. Preferably, the canister and the catheter are blow molded or otherwise formed in a one piece construction. An inlet port on the housing and an outlet chamber from the canister are provided with seals to form an air tight connection between the housing and the canister. The canister is constructed to form a labyrinthine path from the catheter to the outlet chamber.

### CROSS-REFERENCE TO RELATED APPLICATIONS

This application is a continuation-in-part of application Ser. No. 10/376,496 filed Feb. 28, 2003, which claims the benefit U.S. Provisional Application No. 60/360,289 filed Feb. 28, 2002, the disclosures of which are incorporated fully herein by reference.

### BACKGROUND OF THE INVENTION

This invention relates to emergency medical equipment, and more particularly, to a portable battery operated aspirator.

Aspirators are used in an emergency medical setting, usually at the site of a medical emergency or in a hospital, to clear obstructions, namely fluids and debris, from the airway of a patient. Light weight, ease of handling, dependability, exceptional performance, and durability are useful characteristics for this purpose.

### SUMMARY OF THE INVENTION

According to the invention, a portable aspirator has a canister, a housing that removably receives the canister, and a catheter directly connected to the canister. Preferably, the canister and the catheter are molded or otherwise formed in a one piece construction. An inlet port that extends from the housing and an outlet chamber in the canister are provided with seals to form an air tight connection between the housing and the canister. The canister is constructed to form a labyrinthine path from the catheter to the outlet chamber.

### BRIEF DESCRIPTION OF THE DRAWINGS

The features of a specific embodiment of the best mode contemplated of carrying out the invention is illustrated in the drawings, in which:

FIG. 1 is a side elevation view of an aspirator housing and a removable canister incorporating principles of the invention; the canister is removed from the housing;

FIG. 2 is a side elevation view of the canister of FIG. 1 installed in the housing of FIG. 1;

FIG. 3 is an enlarged view of the interface between the housing and canister of FIG. 2;

FIG. 4 is an end view of the canister of FIG.1 taken in the direction of the arrows on the plane 4-4 in FIG. 1;

FIG. 5 is an end view of the housing of FIG. 1 taken in the direction of the arrows on the plane 5-5 in FIG. 1;

FIG. 6 is a top elevation view of the housing and canister of FIG. 2 positioned for shipment or storage when the aspirator is out of use;

FIG. 7 is a side elevation view of the housing and canister of FIG. 2 with carrying straps illustrating how the aspirator may be gripped in use;

FIG. 8 is a front elevation view of the housing and canister of FIG. 2 with the outboard catheter holder cut away to show the finger gripping surface of the PC Board holder along the side of the housing; and

FIG. 9 is a side sectional view of the pressure relief vent.

### DETAILED DESCRIPTION OF THE SPECIFIC EMBODIMENT

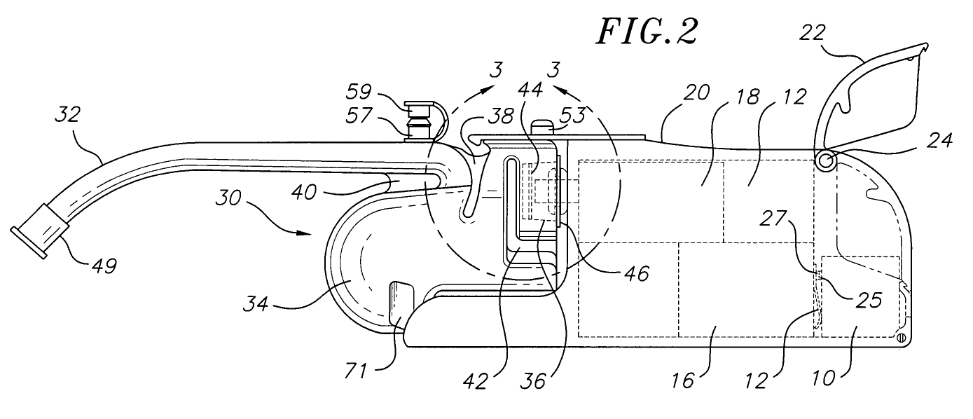

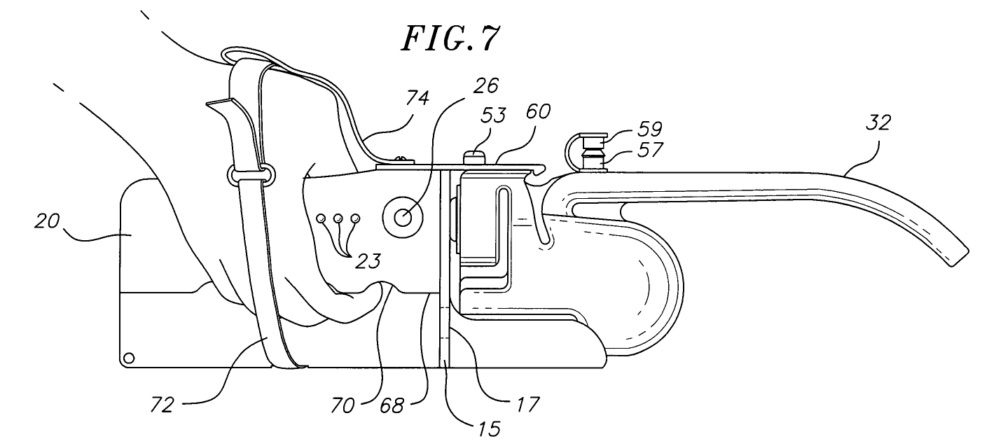

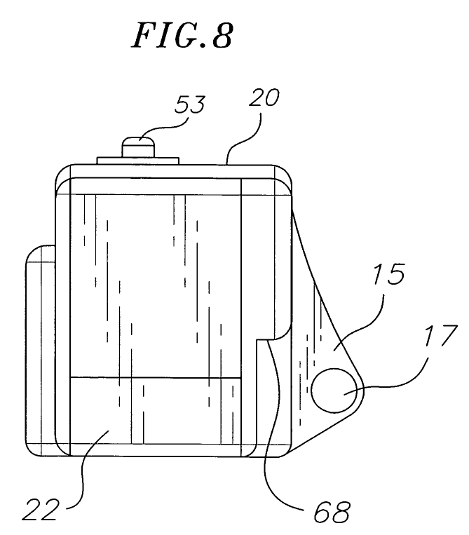

With reference to FIG.2 a removable battery 10, a circuit board 12, an electric motor 16, and a vacuum pump 18 are disposed in a housing 20. Battery 10 has a pair of opposite polarity connecting pads 25 that made electrical contact with a matching pair of opposite polarity connecting pads 27 on circuit board 12 to furnish power thereto. From circuit board 12 power is delivered to motor 16, which drives pump 18. Electronics for performing various functions such as battery charging and status display are mounted on circuit board 12. Battery 10 is charged through an external receptacle (not shown). Status lamps 23 are displayed through housing 20 (FIG. 7). An outboard catheter holder 15 with a hole 17 is formed in the side of housing 20 (FIG. 5). An intake port 19 of the pump 18 protrudes through housing 20. An access door 22 at the back of housing 20 has a hinge 24. The unhinged end of door 22 is designed to form a snap fit with housing 20 and thereby compress one of the pairs of pads is compressed when door 22 is closed to insure electrical contact between battery 10 and circuit board 12. To replace battery 10, door 22 is swung open as shown in FIG. 2 and free pads 25 and 27 so battery 10 can be removed. A start button 26 (FIG. 7) that is accessible from the exterior of housing 20 turns motor 16 on and off.

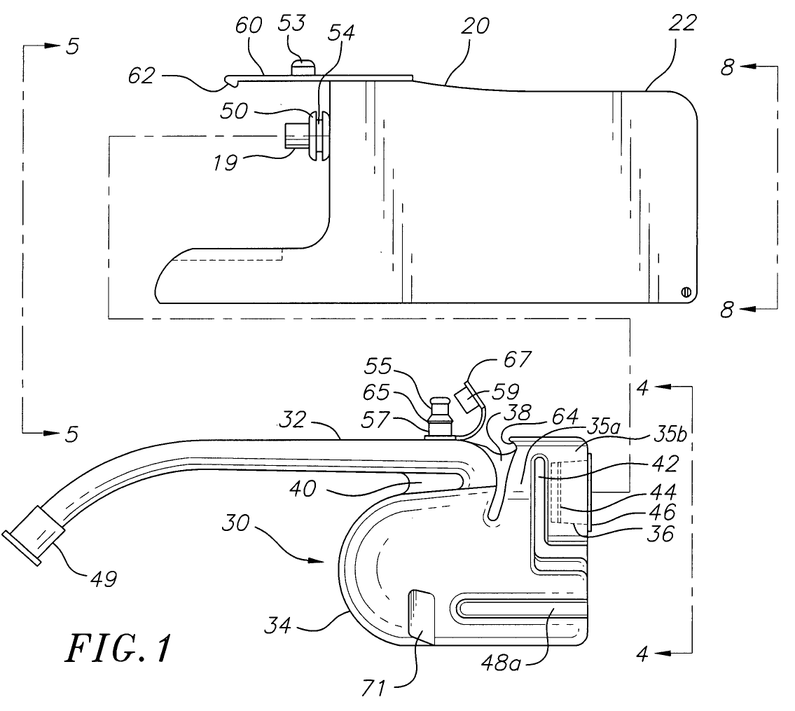

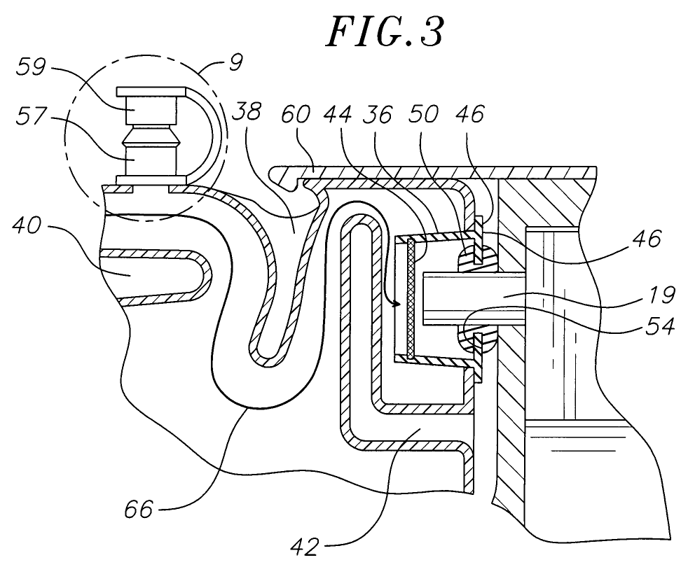

With particular reference to FIG. 2, a disposable canister 30 is preferably molded or vacuformed from clear non-reactive plastic, such as polypropylene. In contrast to housing 20, canister 30 is designed for single use. A catheter 32, which has a downwardly curved anatomically correct shape for insertion in the throat of a human patient, is directly connected to canister 30. (Catheter 32 is preferably rigid.) As a result of the shortened flow path, there is little flow resistance between the catheter and canister, which permits the use of a small motor and pump. The sharp edge formed on the end of catheter 32 by the molding process is rounded by heating it with ultrasonic or radio frequency energy. The end of catheter 32 is also provided with pressure relief holes (not shown) to keep the high suction away from the surface of the patient. Catheter 32 is molded with canister 30 in a one piece construction. Alternatively, however, catheter 32 could be formed as a separate part that is coupled to canister 30 by, for example, a threaded or bayonet connection. Catheter 32 feeds a collection chamber 34 in canister 30, which leads to a slightly conical outlet chamber 36. Webs 38, 40, and 42 are formed in the molding process. Web 38 forms a goose neck-like path between catheter 32 and collection chamber 34 and web 42 forms a constricted path from collection chamber 34 to outlet chamber 36. As shown in FIGS. 1 and 2, a detent 71 is formed on the outer surface of collection chamber 34 for the purpose of retaining canister 30 during storage. Outlet chamber 36 is made from an elastomeric material. A circular filter 44 is mounted in outlet chamber 36; for example by a groove that captures the edge of filter 44, and an annular elastomeric sealing washer 46 extends around the exit from outlet chamber 36 (FIG. 1). Filter 44 is a porous hydrophobic disk coated with a material that blocks the pores when exposed to water. An exemplary filter for this purpose is Porex part no. X-5422. An elastomeric grommet 50 is mounted on intake port 19 (FIG. 1). A groove 54, is formed around the periphery of grommet. As shown in FIG. 3, groove 54 is captured by the edge at the inner diameter of washer 36.

A pressure relief vent 55 is formed on catheter 32 (FIG. 9). Vent 55 protrudes from catheter 32 as a boss 57 so a tethered cap 59 can fit thereover. Cap 59 has a tether 61 with a loop 63. Loop 63 fits over boss 57 where it is captured by a retaining ring 65 on boss 57. Loop 63 has enough elasticity to pass over ring 65 as it is captured. When vent 55 is closed by cap 59, a high vacuum (e.g., 500 mmHg) suitable for adult use is created at the inlet of catheter 32 and when vent 55 is open, a low vacuum (e.g., 125 mmHg) suitable for child use is created at the inlet of catheter 32. A flange 67 is formed on the base of cap 59 to enable easy removal of cap 59 when a low pressure at the inlet of catheter 32 is desired. Vent 55 is located near the junction of catheter 32 with canister 30 so it can be accessed by the index finger of an EMT when the aspirator is in use. Thus the EMT can control the inlet pressure by blocking and unblocking vent 55 with his/her index finger. Cap 59 and tether 61 form a one piece construction. Because of the proximity of vent 55 to holding straps 72 and 74 (FIG. 7), the EMT can open and close the vent with the index finger of the same hand that grips the aspirator. In summary, vent 55 and cap 59 function together as a convenient pressure adjustment for the aspirator.

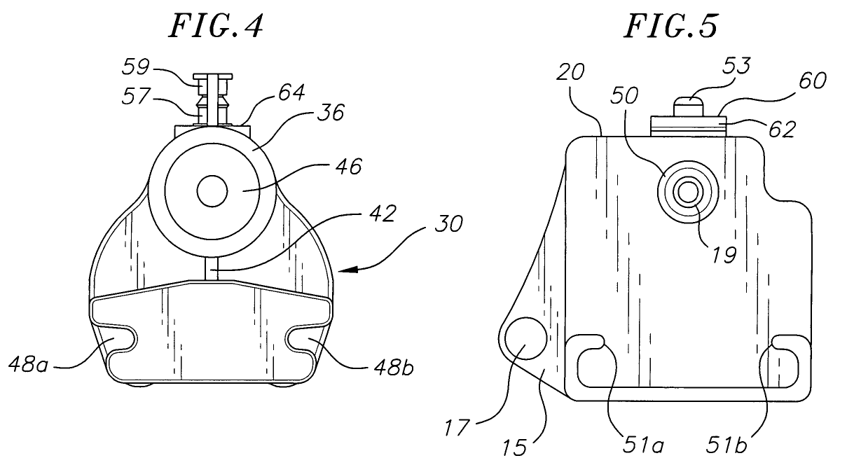

To install canister 30 on housing 20, side grooves 48a , 48b on canister 30 (FIG. 4) slide on rails 51a,51b toward housing 20 so sealing washer 46 is aligned with intake port 19 until sealing washer 46 abuts and compresses grommet 50 to insure an air and liquid tight connection to housing 20. Grommet 50, which abuts the front surface of housing 20, serves as a stop on the movement of canister 30 during installation. As a result, washer 46 and grommet 50 are both somewhat compressed, which insures that no leakage takes place at the interface of canister 30 and housing 20. A resilient clamp 60 mounted on housing 20 has a downwardly facing hook 62 that snaps against a lip 64 on the top of canister 30 as canister 30 is installed. Clamp 60 holds canister 30 against housing 20 so washer 46 and grommet 50 remain compressed. To disengage canister 30 from housing 20, clamp 60 is pushed upwardly, which releases hook 62 from lip 64.

When the aspirator is turned on by start button 26, suction is developed at the end of catheter 32. Consequently, air and entrained bodily fluids are drawn through catheter 32 into collection chamber 34. As shown by a line 66 (FIG. 3), the air follows a labyrinthine path from catheter 32 to outlet chamber 36, but the fluids are by in large deposited in chamber 34. The, air and entrained fluids are first drawn downwardly due to the baffle formed by web 38, where much of the fluid is separated from the air. The air then flows up and around the baffle formed by web 42 and into filter 44. There is a narrow space between the baffle formed by web 42 and the end of outlet chamber 36 restricting the access of fluids to the surface of filter 44. In addition, any fluids that reach the surface of filter 44 are prevented from entering intake 19 because of the characteristics of filter 44 described above. In summary, the labyrinthine path separates, to a large degree, the bodily fluids from the airflow, and thus reduces the exposure of the filter element 44 to the bodily fluids, and filter 44 provides further assurance that pump 18 remains free of such fluids. A removable cap 49 is stored on a peg 53 (FIG. 5) on clamp 60 when the aspirator is turned on. After the aspirator is turned off, cap 49 is placed on the end of catheter 32 to prevent escape of fluids from canister 30.

For a right-handed user, the right side of housing 20 has a longitudinal shelf 68 with finger grips 70 along its edge. A strap 72 extends loosely enough around housing 20 for insertion of a user's hand. Strap 72 has a cross-strap 74 which is secured to clamp 60. FIG. 7 illustrates how the user may hold the aspirator in use.

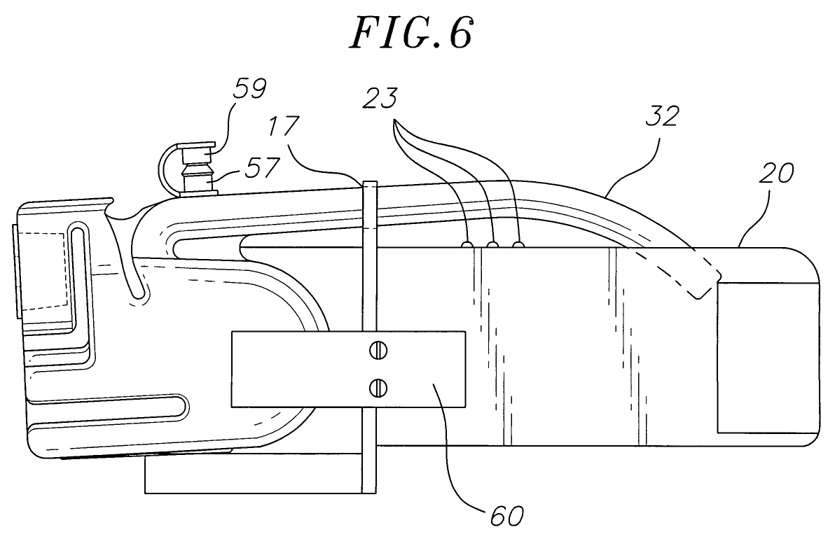

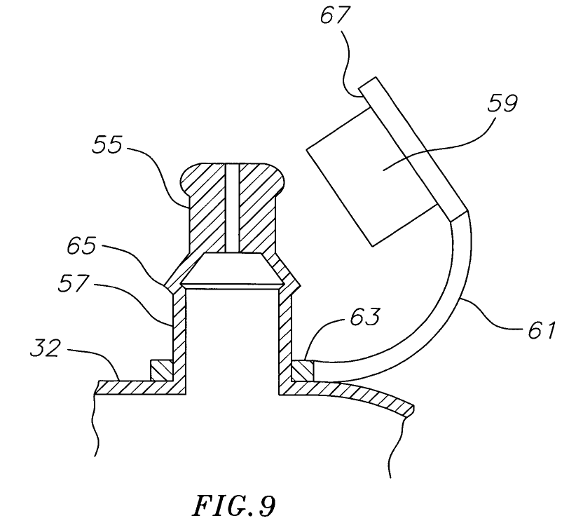

To store or transport the described aspirator, canister 30 is flipped around 180 degrees so catheter 32 faces housing 20, then turned 90 degrees from its installed orientation, and pushed into hole 17 in holder 15 (FIG. 8) where it is secured by detent 71 (see FIG. 1) when it engages clamp 60 in the storage position shown in FIG. 6. Canister 30 approaches hole from the side so its path of entry into the storage position follows the curve of catheter 32 into hole 17. Alternatively, the storage configuration could be disposed on the other side of the aspirator. As illustrated in FIG. 6, the resulting storage configuration is compact and suitable for shipping to customers and storage between uses by an EMT.

Attached as appendix A is a TRAINING BULLETIN and as Appendix B is OPERATING INSTRUCTIONS & MAINTENANCE MANUAL for the invention.

The described embodiment of the invention is only considered to be preferred and illustrative of the inventive concept; the scope of the invention is not to be restricted to such embodiment. Various and numerous other arrangements may be devised by one skilled in the art without departing from the spirit and scope of this invention.

1. A canister for use in a aspirator comprising:
a fluid collection chamber;
a catheter directly connected to the collection chamber; and
an outlet chamber connected to the collection chamber.

2. The canister of claim 1, in which the fluid collection chamber and the catheter are formed in a one-piece construction.

3. The canister of claim 1, designed for single use.

4. The canister of claim 1, in which a hydrophobic filter is disposed in the outlet chamber.

5. The canister of claim 2, in which the catheter has a downwardly curved anatomically correct shape for insertion in the throat of a human patient.

6. The canister of claim 3, in which the canister has an exit and an annular seal that surrounds the exit to form an air tight connection to the intake port of the housing.

7. The canister of claim 1, in which canister is constructed to form a labyrinthine path from the catheter to the intake port of the housing.

8. The canister of claim 1, in which the canister is constructed so the canister has a collection chamber near the bottom of the canister, the catheter is near the top of the front of the canister, and the exit is near the top of the back of the canister.

9. The canister of claim 8, in which the outlet chamber has an exit and the canister is constructed to form a rear baffle between the collection chamber and the exit.

10. The canister of claim 9 constructed to form a front baffle that deflects air entering the canister from the catheter toward the bottom of the collection chamber.

11. The canister of claim 1, in which the catheter has a pressure relief vent.

12. The canister of claim 11, in which the vent forms a boss.

13. The canister of claim 12, additionally comprising a cap designed to close the vent.

14. The canister of claim 13, in which the cap is tethered.
